(19) United States
(12) Patent Application Publication
(10) Pub. No.: US 2006/0035251 A1
(43) Pub. Date: Feb. 16, 2006

(54) Novel methods for high-throughput genome-wide location analysis

(75) Inventors: Richard A. Young, Weston, MA (US); Tong Ihn Lee, Cambridge, MA (US); David Reynolds, Allston, MA (US); Tom Volkert, Cambridge, MA (US)

Correspondence Address: FISH & NEAVE IP GROUP;ROPES & GRAY LLP, ONE INTERNATIONAL PLACE, BOSTON, MA, 02110-2624, US

(73) Assignee: Whitehead Institute for Biomedical Research, Cambridge, MA (US)

(21) Appl. No.: 11/171,938

(22) Filed: Jun. 30, 2005

Related U.S. Application Data
(60) Provisional application No. 60/584,904, filed on Jun. 30, 2004.
(60) Provisional application No. 60/634,569, filed on Dec. 9, 2004.

(51) Int. Cl. C12Q 1/68; C12N 1/08; C07H 21/04

(52) U.S. Cl. 435/6; 435/270; 536/25.3

(57) ABSTRACT

The invention relates to improved methods of identifying the genomic regions to which a protein of interest binds, and in particular, to methods that are highly-sensitive and/or high throughput. The invention also provides methods of identifying agents which modulate the binding of a protein to the genome of a cell and methods of identifying variant proteins, such as transcription factors, with altered genome-binding properties. The invention also provides kits related to the methods described herein.

### CROSS-REFERENCE TO RELATED APPLICATIONS

This application claims the benefit of the filing date of U.S. Application No. 60/584904, filed Jun. 30, 2004, entitled “NOVEL METHODS FOR HIGH-THROUGHPUT GENOME-WIDE LOCATION ANALYSIS” and of U.S. Application No. 60/634569, filed Dec. 9, 2004, entitled “NOVEL METHODS FOR HIGH-THROUGHPUT GENOME-WIDE LOCATION ANALYSIS.” The entire teachings of the referenced applications are incorporated by reference herein.

### STATEMENT REGARDING FEDERALLY-SPONSORED RESEARCH OR DEVELOPMENT

The invention described herein was supported, in whole or in part, by the National Institute of Health Grant No. NIH HG002668-01IH. The United States government has certain rights in the invention.

### BACKGROUND OF THE INVENTION

Gene expression is controlled by transcriptional regulatory proteins, which bind specific DNA sequences and recruit cofactors and the transcription apparatus to promoters. The expression of transcriptional regulators themselves is also regulated by transcriptional regulators, and a single gene may be regulated by multiple transcription factors.

Genome-wide analysis methods have been used to determine how tagged transcriptional regulators encoded in Saccharomyces cerevisae are associated with the genome in living yeast cells and to model the transcriptional regulatory circuitry of these cells. These methods have also been used in human tissue culture celis to identify target genes for several transcriptional regulators.

However, these methods are low-throughput and labor intensive. A need remains to develop efficient genome-scale analysis methods that allow high-throughput analysis of transcriptional regulators. A need also remains to develop sensitive genome-wide analysis methods to elucidate the global gene expression programs that characterize specific tissues, and in particular, freshly isolated, primary tissues of limited availability. Furthermore, there is a need for genome-scale high-throughput, high-content analysis methods that enable the screening and identification of agents that modulate the activity of transcriptional regulators in a cell. The present invention provides these and other methods.

### SUMMARY OF THE INVENTION

The present invention relates, in part, to both high-throughput methods and to highly-sensitive methods, and related kits, for identifying a region (one or more) of a genome of a cell to which a protein of interest binds. In one embodiment of the methods described herein, a DNA binding protein of a cell is optionally linked (e.g., covalently crosslinked) to genomic DNA of a cell. The genomic DNA to which the DNA binding protein is linked is identified and combined or contacted with DNA comprising a sequence complementary to genomic DNA of the cell (e.g., all or a portion of a cell's genomic DNA such as one or more chromosome or chromosome region) under conditions in which hybridization between the identified genomic DNA and the sequence complementary to genomic DNA occurs. Region(s) of hybridization are region(s) of the genome of the cell to which the protein of interest binds. The methods of the present invention are preferably performed using live cells, but may also be performed using preserved or fixed cells.

The invention provides methods that can be used to examine and/or identify proteins across the entire genome of an eukaryotic organism. For example, DNA binding proteins across the entire genome of eukaryotic organisms such as yeast, Drosophila and humans can be analyzed. Alternatively, they can be used to examine and/or identify DNA binding of proteins to an entire chromosome or set of chromosomes of interest. Similarly, a variety of proteins which bind to DNA can be analyzed. For example, any protein involved in DNA replication such as a transcription factor, or an oncoprotein can be examined in the methods of the present invention. In addition, the high sensitivity of the methods provided herein allows genome-wide location analysis to be performed using limited numbers of cells, and in particular, limited numbers of primary human cells. The invention also provides kits, including kits that facilitate the isolation of DNA from cells suitable for location analysis.

### DETAILED DESCRIPTION OF THE INVENTION

### I. OVERVIEW

The invention provides, in part, novel high-throughput and highly-sensitive methods for the identification of protein-DNA interactions in a cell. The high-throughput methods described herein allow the identification of the DNA regions to which a protein is bound under different conditions, such as when the cell is contacted with an experimental agent. The invention also provides methods of identifying the genomic regions to which a protein is bound, using a very small number of cells or genomic equivalents. Such improvements in sensitivity enable chromosome-wide location analysis to be used as a diagnostic tool, such as in diagnosing disease states from samples of tissue biopsies, or as a drug discovery tool.

One aspect of the invention provides methods for identifying a region or regions of the genome of a cell to which a protein of interest is directly or indirectly bound. One aspect of the invention provides a method for identifying a region of a genome of a cell to which a protein of interest is bound, the method comprising the steps of: (a) fragmenting the genomic DNA of the cell by: (i) a mechanical or chemical process; and (ii) by an enzymatic means, thereby producing a mixture comprising DNA fragments to which the protein of interest is bound; (b) isolating a DNA fragment to which the protein of interest is bound from the mixture produced in step (a); and (c) identifying a region of the genome of the cell which is complementary to the DNA fragment isolated in step (b), thereby identifying a region of a genome of a cell to which the protein of interest is bound.

Another aspect of the invention provides a method for identifying a region of a genome of a cell to which a protein of interest is bound, the method comprising the steps of: (a) crosslinking the protein of interest to genomic DNA of a population of cells, thereby producing protein of interest crosslinked to genomic DNA; (b) fragmenting the genomic DNA crosslinked to the protein of interest by: (i) a mechanical or chemical process; and (ii) by enzymatic means thereby producing a mixture comprising DNA fragments to which protein of interest is bound; (c) removing a DNA fragment to which the protein of interest is bound from the mixture produced in (b); (d) separating the DNA fragment identified in (c) from the protein of interest; (e) generating labeled probes from the fragment generated in (d) by using the fragment as a template for DNA synthesis by DNA polymerase, wherein the DNA synthesis is primed using random primers; (f) combining the labeled probes with DNA comprising a sequence complementary to genomic DNA of the cell, under conditions in which hybridization between the DNA fragment and a region of the sequence complementary to genomic DNA occurs; and (g) identifying the region of the sequence complementary to genomic DNA of (f) to which the DNA fragment hybridizes, whereby the region identified in (g) is the region of the genome in the cell to which the protein of interest is bound.

The invention also provides methods of identifying transcription factors whose activity underlies the developmental program of a cell, such as a stem cell. One specific aspect of the invention provides a method of identifying the region of a genome of a stem cell to which a protein of interest is bound during differentiation of the stem cell, the method comprising (a) culturing the cell under conditions that promote the differentiation of the cell; and (b) identifying the region of a genome of a stem cell to which a protein of interest is bound, according to any of the methods described herein, wherein the protein of interest is a transcriptional regulator. Conditions which promote the differentiation of a stem cell will vary according to the stem cell. In some embodiments, the conditions for inducing differentiation comprise contacting the cell with a growth factor or other secreted protein and/or culturing the stem cell with one or more additional cell types. The stem cells may be cultured as single cells or as part of tissues or even organs.

In other embodiments, rather than culturing the stem cell ex-vivo, the cell is in an organism, such as in an animal. When the cell is in an organism, identifying the region of the genome to which the protein of interest is bound may be effected at a time when the cell is expected to undergo differentiation, such as at a particular stage of development. Alternatively, the organism may be treated with an agent, such as a small molecule drug or an RNAi nucleic acid, which promotes or blocks the differentiation of the stem cell. These methods may be useful for identifying agents which promote or block the differentiation of stem cells, which in turn may be useful in the development of therapeutics.

In one embodiment, the stem cell is selected from the group consisting of an embryonic stem cell, a placental stem cell, adult stem cell, partially differentiated stem cell, cord blood stem cell, a peripheral blood stem cell, and a bone marrow stem cell. The stem cell may be a germline stem cells or a somatic stem cells. In a specific embodiment, the stem cell is selected from the group consisting of embryonic stem cells, somatic stem cells, germ stem cells, epidermal stem cells, adult neural stem cells, keratinocyte stem cells, melanocyte stem cells, adult renal stem cells, embryonic renal epithelial stem cells, embryonic endodermal stem cells, hepatocyte stem cells, mammary epithelial stem cells, bane marrow-derived stem cells, skeletal muscle stem cells, bone marrow mesenchymal stem cells, CD34+ hematopoietic stem cells and mesenchymal stem cells.

The invention also provides methods for identifying or screening mutant transcriptional regulators. The high-throughput, highly-sensitive genome-wide location analysis methods of the present invention allow the screening of large numbers of mutant transcriptional regulators, including those from mutant libraries generated by in vitro or in vivo mutagenesis. The methods are also useful to screen transcription factor drugs for minimal side effects by selecting mutants showing minimal binding to nontarget promoters.

For example, a transcriptional regulator may be designed which binds to a given sequence using methods known in the art such as those described in U.S. Pat. Nos. 6,607,882, 6,453,242 and 6,511,808. A panel of mutant transcription factors can then be generated by mutagenic PCR techniques, transfected into cells to generate a library, and library members screened to identify the genomic sites to which the mutant transcriptional regulator is bound using the methods described herein. Those transcriptional regulators which display minimal binding to unwanted sites may be selected.

On specific aspect of the invention provides a method of screening a panel of mutant transcriptional regulators for regulators which bind to a specific set of regions of a genome in a cell, the method comprising (a) expressing each transcriptional regulator in a cell; (b) identifying regions of the genome of the cell to which the transcriptional regulators bind using any of the methods described herein, and (c) comparing the regions of the genome of the cell to which each transcriptional regulator binds to the specific set of regions and (d) selecting those transcriptional regulators which bind to the specific set of regions.

The high-throughput methods described herein may be used to screen and identify mutant forms of transcriptional regulators having an altered activity. In a specific embodiment, the altered activity in the transcriptional regulator comprises at least one of the following: (a) an alteration in the binding affinity of the transcriptional regulator to DNA; (b) an alteration in the ability of the transcriptional regulator to bind to RNA polymerase, to an RNA polymerase holoenzyme, or to a second transcriptional regulator; (c) an alteration in the binding affinity of the transcriptional regulator to a ligand; (d) an alteration in expression level or expression pattern of the transcriptional regulator; or (e) an alteration in an ability of the transcriptional regulator to form homomultimers or heteromultimers. It is well-known to one skilled in the art that mutations in a transcriptional regulator may result in a hypomorphic, hypermorphic or neomorphic phenotype. Mutations may generally reduce the activity of a transcriptional regulator, may generally increase its activity, or may confer novel properties, such as altering its range of targets or turning it from an activator into a repressor or vice versa. A cell expressing a transcriptional regulator having any of these alterations in activity may be used.

### II. DEFINITIONS

For convenience, certain terms employed in the specification, examples, and appended claims, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

A “patient” or “subject” to be treated by the method of the invention can mean either a human or non-human animal, preferably a mammal.

The term “encoding” comprises an RNA product resulting from transcription of a DNA molecule, a protein resulting from the translation of an RNA molecule, or a protein resulting from the transcription of a DNA molecule and the subsequent translation of the RNA product.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, protein or both.

“Recombinant” when used with-reference, e.g., to a nucleic acid, cell, virus, plasmid, vector, or the like, indicates that these have been modified by the introduction of an exogenous, non-native nucleic acid or the alteration of a native nucleic acid, or have been derived from a recombinant nucleic acid, cell, virus, plasmid, or vector. Recombinant protein refers to a protein derived from a recombinant nucleic acid, virus, plasmid, vector, or the like.

The term “transcriptional regulator” refers to a biochemical element that acts to prevent or inhibit the transcription of a promoter-driven DNA sequence under certain environmental conditions (e.g., a repressor or nuclear inhibitory protein), or to permit or stimulate the transcription of the promoter-driven DNA sequence under certain environmental conditions (e.g., an inducer or an enhancer).

The term “microarray” refers to an array of distinct polynucleotides or oligonucleotides synthesized on a substrate, such as paper, nylon or other type of membrane, filter, chip, glass slide, or any other suitable solid support.

The terms “disorders” and “diseases” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

The terms “level of expression of a gene in a cell” or “gene expression level” refer to the level of mRNA, as well as pre-mRNA nascent transcript(s), transcript processing intermediates, mature mRNA(s) and degradation products, encoded by the gene in the cell.

The term “modulation” refers to upregulation (i.e., activation or stimulation), downregulation (i.e., inhibition or suppression) of a response, or the two in combination or apart. A “modulator” is a compound or molecule that modulates, and may be, e.g., an agonist, antagonist, activator, stimulator, suppressor, or inhibitor.

The term “agonist” refers to an agent that mimics or up-regulates (e.g., potentiates or supplements) the bioactivity of a protein, e.g., polypeptide X. An agonist may be a wild-type protein or derivative thereof having at least one bioactivity of the wild-type protein. An agonist may also be a compound that upregulates expression of a gene or which increases at least one bioactivity of a protein. An agonist may also be a compound which increases the interaction of a polypeptide with another molecule, e.g., a target peptide or nucleic acid.

The term “antagonist” refers to an agent that downregulates (e.g., suppresses or inhibits) at least one bioactivity of a protein. An antagonist may be a compound which inhibits or decreases the interaction between a protein and another molecule, e.g., a target peptide or enzyme substrate. An antagonist may also be a compound that downregulates expression of a gene or which reduces the amount of expressed protein present.

The term “prophylactic” or “therapeutic” treatment refers to administration to the subject of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “therapeutic effect” refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically-effective amount of a compound will depend on its therapeutic index, solubility, and the like. For example, certain compounds discovered by the methods of the present invention may be administered in a sufficient amount to produce a reasonable benefit/risk ratio applicable to such treatment.

A probe that is “labeled” is detectable, either directly or indirectly, by spectroscopic, photochemical, biochemical, immunochemical, isotopic, or chemical means. For example, useful labels include ³²P, ³³P, ³⁵S, ¹⁴C. ³H, ¹²⁵I, stable isotopes, fluorescent dyes and fluorettes (Rozinov and Nolan (1998) Chem. Biol 5:713-728; Molecular Probes, Inc. (2003) Catalogue, Molecular Probes, Eugene Oreg.), electron-dense reagents, enzymes and/or substrates, e.g., as used in enzyme-linked immunoassays as with those using alkaline phosphatase or horse radish peroxidase. The label or detectable moiety is typically bound, either covalently, through a linker or chemical bound, or through ionic, van der Waals or hydrogen bonds to the molecule to be detected. “Radiolabeled” refers to a compound to which a radioisotope has been attached through covalent or non-covalent means. A “fluorophore” is a compound or moiety that absorbs radiant energy of one wavelength and emits radiant energy of a second, longer wavelength.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe can be detected by detecting the presence of the label bound to the probe. The probes are preferably directly labeled as with isotopes, chromophores, fluorophores, chromogens, or indirectly labeled such as with biotin to which a streptavidin complex or avidin complex can later bind.

A “nucleic acid probe” is a nucleic acid capable of binding to a target nucleic acid of complementary sequence, usually through complementary base pairing, e.g., through hydrogen bond formation. A probe may include natural, e.g., A, G, C, or T, or modified bases, e.g., 7-deazaguanosine, inosine, etc. The bases in a probe can be joined by a linkage other than a phosphodiester bond. Probes can be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions.

“Polymerase chain reaction” (PCR) refers, e.g., to a procedure or product where a specific region or segment of a nucleic acid is amplified, and where the segment is bracketed by primers used by DNA polymerase (Bernard and Wittwer (2002). Clin. Chem. 48: 1178-1185; Joyce (2002) Methods Mol. Biol. 193:83-92; Ong and Irvine (2002) Hematol. 7:59-67).

A “promoter” is a nucleic acid sequence that directs transcription of a nucleic acid. A promoter includes nucleic acid sequences near the start site of transcription, e.g., a TATA box, see, e.g., Butler and Kadonaga (2002) Genes Dev. 16:2583-2592; Georgel (2002) Biochem. Cell Biol. 80:295-300. A promoter also optionally includes distal enhancer or repressor elements, which can be located as much as several thousand base pairs on either side from the start site of transcription. A “constitutive” promoter is a promoter that is active under most environmental and developmental conditions, while an “inducible”, promoter is a promoter is active or activated under, e.g., specific environmental or developmental conditions.

“Small molecule” is defined as a molecule with a molecular weight that is less than 10 kD, typically less than 2 kD, and preferably less than 1 kDa. Small molecules include, but are not limited to, inorganic molecules, organic molecules, organic molecules containing an inorganic component, molecules comprising a radioactive atom, synthetic molecules, peptide mimetics; and antibody mimetics. As a therapeutic, a small molecule may be more permeable to cells, less susceptible to degradation, and less apt to elicit an immune response than large molecules. Small molecule toxins are described, see, e.g., U.S. Pat. No. 6,326,482 issued to Stewart, et al.

### III. METHODS OF IDENTIFYING CHROMOSOMES REGIONS

One aspect of the invention provides methods for identifying a region of a genome of a cell to which a protein of interest is bound. One specific aspect of the invention provides a method for identifying a region of a genome of a cell to which a protein of interest is bound, the method comprising the steps of: (a) fragmenting the genomic DNA of the cell by: (i) a mechanical or chemical process; and (ii) by an enzymatic means, thereby producing a mixture comprising DNA fragments to which the protein of interest is bound; (b) isolating a DNA fragment to which the protein of interest is bound from the mixture produced in step (a); and (c) identifying a region of the genome of the cell which is complementary to the DNA fragment isolated in step (b), thereby identifying a region of a genome of a cell to which the protein of interest is bound.

In one preferred embodiment of the method recited above, step (c) comprises (i) generating a labeled probe from the DNA fragment isolated in step (b); and (ii) combining the labeled probe with at least one nucleic acid comprising a sequence complementary to a region of the genome of the cell, under conditions in which hybridization between the labeled probe and the nucleic acid occurs, and detecting said hybridization, wherein hybridization between the labeled probe and the nucleic acid relative to a suitable control indicates that the protein of interest is bound to the region of the genome to which the sequence of the nucleic acid is complementary.

Cells

In one embodiment, fragmenting the genomic DNA of the cell comprises fragmenting the genomic DNA of a population of cells, wherein the population of cells comprises the cell. In one embodiments of the methods described herein, the population of cells comprises less than 10⁸, 10⁷, 10⁶, 10⁵, 10⁴, 10³, 10², 10 or less than 5 cells. In another specific embodiment, the population of cells comprises a single cell. In some embodiments, the population of cells comprises less than 10⁸, 10⁷, 10⁶, 10⁵, 10⁴, 10³, 10², 10, 5 or 2 cells which express the protein of interest, but also comprises cells which do not express the protein of interest. As an illustrative example, a population of cells may comprise ten human primary CD34+ hematopoietic stem cells expressing a protein of interest and 10⁶ cells (e.g. monocytes) which do not express the protein of interest. In another illustrative example, the ten CD34+ cells may be mixed with 10⁶ HEK cells or with 10⁵ HELA cells to be used as “carrier” cells. In one embodiment, the cell population is a population that has been isolated using FACS sorting.

In one embodiment of the methods described herein, the cells are primary cells. Primary cells are isolated from an organism and have undergone minimum passaging in vitro, and thus maintain most of the phenotypic characteristics of cells in the organism. In a specific embodiment, the primary cells are primary cells that have doubled less than 10 times ex vivo.

In some embodiments, the cell is derived from transplant-grade tissue or freshly isolated tissue. In some embodiments, the cell is derived from a tissue biopsy, such as from a subject afflicted with, or suspected of being afflicted with, a disorder. In another embodiment, the cell is isolated from a bodily fluid or bodily secretion, including serum, plasma, saliva, tears, sweat, semen, amniotic fluid, vaginal secretions, nasal secretions, synovial fluid, spinal fluid, blister fluid, bronchoalveolar lavage fluid, ductal lavage, phlegm, pus, stool and intracranial fluid. The cell may be a live cell or a cell that has been preserved, such as by treatment with formalin, B5, Zenker's fixatives, Lugol's solution, Carnoy's Fixative, F13 fixative, or other preservatives, or a cell that has been preserved by freezing.

The cell type used in the assays described herein may be any cell type. The cell may be an eukaryotic or a prokaryotic cell, from a metazoan or from a single-celled organism such as yeast. In some preferred embodiments, the cell is a mammalian cell, such as a cell from a rodent, a primate or a human. The cell may be a wild-type cell or a cell that has been genetically modified by recombinant means or by exposure to mutagens. The cell may be a transformed cell or an immortalized cell. In some embodiments, the cell is from an organism afflicted by a disease. In some embodiments, the cell comprises a genetic mutation that results in disease, such as in a hyperplastic condition.

In some embodiments of the methods described herein, the cell expresses a mutant form of a transcriptional regulator. A preferred mutant form of the transcriptional regulator is one that causes the disease to which the therapeutic is sought. Such embodiments are particularly preferred when a mutant transcriptional regulator which causes at least one form of the disease has an altered target specificity and thus the genes it regulates, or the extent to which it regulates their transcription, is altered when compared to the non-mutant form of the transcriptional regulator. Such embodiments may allow the identification of agents which restore wild-type activity to mutant transcriptional regulator. Mutations in the DNA binding domain, for example, may alter the target specificity of a transcriptional regulator by altering its affinity for various DNA binding sequences.

Crosslinking/Removing Crosslink

In one embodiment of the methods described herein, the protein of interest is covalently crosslinked to the genomic DNA prior to fragmenting the genomic DNA. There are a variety of methods which can be used to link a DNA-binding to genomic DNA. In one embodiment of the methods described herein, the crosslinking is formaldehyde crosslinking (Solomon, M. J. and Varshavsky, A., Proc. Natl. Sci. USA 82:6470-6474; Orlando, V., TIBS, 25:99-104). UV light may also be used (Pashev et al. Trends Biochem Sci. 1991;16(9):323-6; Zhang L et al. Biochem Biophys Res Commun. 2004;322(3):705-11).

In one embodiment of the methods described herein where the protein of interest is covalently crosslinked to the genomic DNA prior to fragmenting the genomic DNA of the cell, separating the DNA fragment from the protein of interest comprises the step of reversing the crosslink. In a specific embodiment, it comprises the steps of (i) isolating a DNA fragment to which the protein of interest is bound from the mixture produced in (a); and (ii) isolating (1) the DNA fragment from (2) the protein of interest. In a specific embodiment, isolating the DNA fragment from the protein of interest to which it is bound comprises the steps of (1) removing the crosslink between the DNA fragment and the protein of interest; (2) treating the DNA fragment with an RNA-degrading enzyme; (3) treating the DNA fragment with a protease; and (4) purifying the DNA fragment. In a preferred embodiment, step (2) is performed before step (3).

Suitable non-limiting methods for purifying the DNA fragment include column chromatography (U.S. Pat. No. 5,707,812), the use of hydroxylated silica polymers (U.S. Pat. No. 5,693,785), rehydrated silica gel (U.S. Pat. No. 4,923,978), boronated silicates (U.S. Pat. No. 5,674,997), modified glass fiber membranes (U.S. Pat. Nos. 5,650,506; 5,438,127), fluorinated adsorbents (U.S. Pat. No. 5,625,054; U.S. Pat. No. 5,438,129), diatomaceous earth (U.S. Pat. No. 5,075,430), dialysis (U.S. Pat. No. 4,921,952), gel polymers (U.S. Pat. No. 5,106,966) and the use of chaotropic compounds with DNA binding reagents (U.S. Pat. No. 5,234,809). Commercially available DNA isolation and purification kits are also available from several sources including Stratagene (CLEARCUT Miniprep Kit), and Life Technologies (GLASSMAX DNA Isolation Systems).

When treating the DNA fragment with a protease, the protease may be selected from the group consisting of aspartic proteases, serine proteases, thiol proteases, metallo proteases, acid proteases and alkaline proteases. In another embodiment, the protease is a serine protease such as thrombin, plasmin, factor Xa, uPA, tPA, granzyme B, trypsin, chymotrypsin, human neutrophil elastase, or a cysteine protease such as papain and cruzain. In a preferred embodiment, the protease is proteinase K. A mixture of proteases may also be used.

In some embodiments of the methods described herein, the RNA-degrading enzyme is an RNase, such as an RNase exonuclease. In some embodiments of the methods described herein, the RNase is an endonuclease such as RNase E. In another embodiment, the RNase is selected from the group consisting of: RNase A, RNase H, RNase One, RNase B, RNase T1, RNase T2, RNase S, RNase from chicken liver, RNase from Aspergillus clavatus, and pancreatic RNase. In another embodiment, rather than, or in addition to, treating the DNA fragment with an RNA-degrading enzyme, the RNA is degraded using chemical means, such as by heating or alkaline hydrolysis.

Mechanical/Chemical/Enzymatic Fragmentation

In some embodiments of the methods described herein, the mechanical process for fragmenting the genomic DNA comprises hydrodynamic shearing or sonication. Mechanical fragmentation can occur by any method known in the art, including shearing of DNA by passing it through the narrow capillary or orifice (Oefner et al., 1996, Nucleic Acids Res.;24(20):3879-86; Thorstenson et al., 1998, Genome Res.;8(8):848-55), sonicating the DNA, such as by ultrasound (Bankier, 1993, Methods Mol Biol.;23:47-50, or grinding in cell homogenizers (Rodriguez L V. Arch Biochem Biophys. 1980;200(1): 116-29). Mechanical fragmentation usually results in double strand breaks within the DNA molecule. Sonication may also be performed with a tip sonicator, such as a multi-tip sonicator, or more preferably using acoustic soundwaves. A Microplate Sonicator® (Misonix Inc.) may be used to partially fragment the DNA. Such a device is described in U.S. patent Publication No. 2002/0068872. Another acoustic-based system that may be used to fragment DNA is described in U.S. Pat. No. 6,719,449, manufactured by Covaris Inc. U.S. Pat No. 6,235,501 describes a mechanical method of producing high molecular weight DNA fragments by application of rapidly oscillating reciprocal mechanical energy to cells in the presence of a liquid medium in a closed container, which may be used to mechanically fragment the DNA.

In some embodiments of the methods described herein, the chemical process for fragmenting DNA comprises acid catalytic hydrolysis, alkaline catalytic hydrolysis, hydrolysis by metal ions, hydroxyl radicals or irradiation. Chemical fragmentation of DNA can be achieved by any method known in the art, including acid or alkaline catalytic hydrolysis of DNA (Richards and Boyer, 1965), hydrolysis by metal ions and complexes (Komiyama and Sumaoka, 1998; Franklin, 2001; Branum et al., 2001), hydroxyl radicals (Tullius, 1991; Price and Tullius, 1992) or radiation treatment of DNA (Roots et al., 1989; Hayes et al., 1990). In another embodiment, the chemical process for fragmenting comprises ionizing radiation, such as gamma ray irradiation, X-ray irradiation or combinations thereof. Chemical treatment could result in double or single strand breaks, or both.

In a specific embodiment, the chemical process comprises a manganese porphyrin complex. In a specific embodiment, the porphyrin complex is Mn-TMPyP/KHSO(5) (Chworos A et al. J Biol Inorg Chem. (2004);9(3):374-84). In another specific embodiment, the chemical fragmentation comprises using chemicals which cleave DNA at specific nucleotide bases e.g. piperidine, such as is used in Maxam-Gilbert DNA sequencing (Maxam and W. Gilbert, 1977, Proc. Nat'l. Acad. Sci. USA 74:560).

In one embodiment of the methods described herein, fragmenting the genomic DNA with the mechanical or chemical means generates DNA fragments having an average size of about 2 kb or greater. In some embodiments, the average size of the fragments generated by fragmenting the genomic DNA of the cell is greater than about 3 kb, 4 kb, 5 kb, 6 kb, 7 kb, 8 kb, 9 kb, 10 kb or 15 kb. In a related embodiment of the methods described herein, the mixture comprises DNA fragments having an average size greater than 1, 1.5, 2, 2.5, 3, 4 or 5 kb.

In preferred embodiments of the methods described herein, the genomic DNA is fragmented by a mechanical or chemical process followed by enzymatic digestions. Applicants have made the unexpected discovery that a two step cleavage of genomic DNA yields superior fragmentation to mechanical processes alone. In one specific embodiment, the enzymatic means comprises an endonuclease, such as a restriction enzyme endonuclease. Restriction enzymes which maybe used in the methods described herein include Sfu I (Asu II), Afl III, Bfr I (Afl II), BbrP I (PmaC I), BssH II, Eco47 III, Ecl XI (Xma HI), Hind III, Mam I, Nsp I, Ksp I (Sac II), BstX I, Ita I, Mro I (Acc III), Nar I, Cel II, Cfr10 I, Hind II, Avi II (Aos I), ScrF I (Dsa V), Acy I (Aha II), AspH I (HgiA I), Ksp632 I, Mvn I (FnuD II), Eae I (Cfr I), Sty I, Nde I, EcoR I, Nde II (Mbo I), Not I, Spe I, Fok I, SnaB I, Ssp I, Nsi I, Mlu I, Nhe I, Mae II, Cla I, Dra III, Dra II, Nco I, Dde I, Asp700 (Xmn I), Mae III, Mae I, Asp718, Nae I, Dra I, BstE II, Hinf I, Nru I, Sca I, BpuA I, Aat II, Ban II, Stu I, Dpn I, Bgl II, Xho II, Kpn I, Ava II, Ava I, SspB I, Rsa I, Acc I, Xho I, Apa I, Bgl I, Sau3A I, Hae III, Hae II, Bcl I, Cfo I (Hha I), Xba I, Sac I (Sst I), EcoR V, Sau96 I, BamH I, Pvu I, Pvu II, Msp I, Sph I, Taq I, Sma I, Sal I, XmaC I, BspLU11 I, Bln I (Avr II), Psp1406 I, Acs I, MluN I (Bal I), SexA I, Rca I (BspH I), PinA I (Age I), Pst I, Tru9 I, Alw44 I (Sno I), Mun I (Mfe I), AspE I, EcoR II (BstN I), BseA I, BsiW I, BsiY I, Van91 I (PflM I), Hpa II, Bst1 107 I, Swa I, Meganuclease I-Sce I, Omega Nuclease Omega Transposase, Rsr II, Bsm I, Mva I (BstN I), Sfi I, SgrA I, Bmy I (Bsp1286 I), Hpa I and Alu I. In one specific embodiment, the restriction endonuclease is selected from the group consisting of Sau3a, Styl, NlaIII and Hsp 92. In some embodiments, the enzymatic means comprises a combination of more than one restriction enzyme endonuclease, such as a combination of three or more restriction enzyme endonucleases. In another embodiment, the enzymatic means comprises DNAse I. In some embodiments, the restriction endonucleases recognize 4, 5, 6, 7 or 8 base pair recognition sequences.

Applicants have also made the unexpected discovery that the DNA yields for the foregoing methods are superior when the enzymatic digestions are performed at a reduced temperature, even when such reduced temperature is suboptimal for enzymatic catalysis by the particular enzyme used. The temperature optimum for cleavage of DNA by specific restriction endonucleases that are commercially available is described in the literature, such as in A. Pingoud, Restriction Endonucleases Nucleic Acids and Molecular Biology Series; Springer-Verlag New York, LLC (2004). Thus, in one preferred embodiment, fragmentation by enzymatic means is performed at a reduced temperature relative the optimum temperature.

In a preferred embodiment, enzymes are used which have an optimal temperature for enzymatic activity greater than 20° C., 25° C., 30° C., 35° C., or 37° C., or an optimal temperature of about 37° C. In a specific embodiment, the enzymatic digestions are performed at less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 36 or 37° C. In some embodiments, digestions are performed at about 0° C., or even below 0° C. In a preferred embodiment, the digestions are performed between 4° C. and 20° C., at about 16° C., or at room temperature. In one embodiment, the enzymatic digestions are performed at a temperature that is at least 10° C. below the temperature optimum for the enzyme.

Applicants have also made the unexpected discovery that the DNA yields for the methods described herein are superior when the enzymatic DNA cleavage reactions are performed in solutions that are free, or essentially free, of free amino groups. Accordingly, in one specific embodiment, enzymatic digestion is performed in a solution comprising a buffering agent lacking free amino groups. In preferred embodiments, the solution is free, or essentially free, of Tris i.e. (Tris(Hydroxymethyl) Aminomethane). In a related embodiment, the solution in which the digestion takes places is substantially free of Tris. In a specific embodiment, the concentration of tris in the solution, or of free amino groups, is less than 5 mM, 3 mM, 1 mM, 0.5 mM, 0.3 mM, 0.1 mM, 0.05 mM, 0.01 mM, or 0.001 mM. In one embodiment, the enzymatic reaction is performed in a solution comprising a buffering agent selected from the group consisting of alkali or alkali earth tartrates, alkali or alkali earth carbonates, phosphates, bicarbonates, citrates, borates, acetates, and succinates.

In some embodiments, the enzymatic reaction is performed in a solution comprising a buffering agent selected from the group consisting of HEPES (N-2-hydroxyethylpiperazine-N′-2-ethanesulfonic acid), BES(N,N-bis[2-hydroxyethyl]-2-amino-ethanesulfonic acid), TES(N-tris[Hydroxymethyl]methyl-2-aminoethanesulfonic acid), MOPS (morpholine propanesulphonic acid), PIPES (piperazine-N,N′-bis[2-ethane-sulfonic acid]) and MES (2-morpholino ethanesulphonic acid). In another embodiment, the solution is buffered with sodium citrate/citric acid buffers or phosphate buffers.

Chromatin Immunoprecipitation (ChIP)

In a preferred embodiment, the chromatin fragments bound by the protein of interest (e.g. a transcriptional regulator) are isolated using chromatin immunoprecipitation (ChIP). Briefly, this technique involves the use of a specific antibody to immunoprecipitate chromatin complexes comprising the corresponding antigen i.e. the protein of interest, and examination of the nucleotide sequences present in the immunoprecipitate. Immunoprecipitation of a particular sequence by the antibody is indicative of interaction of the antigen with that sequence. See, for example, O'Neill et al. in Methods in Enzymology, Vol. 274, Academic Press, San Diego, 1999, pp. 189-197; Kuo et al. (1999) Method 19:425-433; and Ausubel et al., supra, Chapter 21. Accordingly, in one embodiment, the DNA fragment bound by the protein of interest is identified using an antibody which binds to the protein of interest.

In one embodiment, the chromatin immunoprecipitation technique is applied as follows. Cells which express the protein of interest, such as a native transcriptional regulator or a recombinant transcriptional regulator, are treated with an agent that crosslinks the transcriptional regulator to chromatin if that transcriptional regulator is stably bound to it. The transcriptional regulator can be crosslinked to chromatin by, for example, formaldehyde treatment or ultraviolet irradiation. Subsequent to crosslinking, cellular nucleic acid is isolated, fragmented and incubated in the presence of an antibody directed against the transcriptional regulator. Antibody-antigen complexes are precipitated, crosslinks are reversed (for example, formaldehyde-induced DNA-protein crosslinks can be reversed by heating) so that the sequence content of the immunoprecipitated DNA is tested for the presence of a specific sequence, for example, promoter regions. The antibody may bind directly to an epitope on the transcriptional regulator or it may bind to a tag on the regulator, such as a myc tag when used with an anti-Myc antibody (Santa Cruz Biotechnology, sc-764). In yet another embodiment, a non-antibody agent with affinity for the transcriptional regulator, or for a tag fused to it, is used in place of the antibody. For example, if the transcriptional regulator comprises a six-histidine tag, complexes may be isolated by affinity chromatography to nickel-containing sepharose. Additional variations on ChIP methods may be found in Kurdistani et al. Methods. 2003 31(1):90-5; O'Neill et al. Methods. 2003, 31(1):76-82; Spencer et al., Methods. 2003;31(1):67-75; and Orlando et al. Methods 11: 205-214 (1997).

In one embodiment of the methods described herein, DNA fragments from a control immunoprecipitation reaction are used in place of the isolated chromatin as a control. For example, an antibody that does not react with a transcription factor being tested may be used in a chromatin IP procedure to isolate control chromatin, which can then be compared to the chromatin isolated using an antibody that binds to the transcriptional regulator. In preferred embodiments, the antibody that does not bind to the transcription factor being tested also does not react with other transcriptional regulators or DNA binding proteins.

Identifying a Genome Region from Isolated DNA Fragments

The identification of genomic regions from the isolated DNA fragments may be achieved by generating DNA or RNA probes from the fragment, and hybridizing them to a DNA microarray, such as a DNA microarray comprising immobilized nucleic acids complementary to regions of the genome of the cell. In some embodiments, the probes themselves are labeled to facilitate their detection. In other embodiments, detection agents may be used to label the DNA/RNA probes once they have hybridized to a DNA microarray. Such detection agents include antibodies, fragments thereof, and dendrimers among others.

A preferred embodiment of the methods described herein comprises generating labeled probes by using the DNA fragments as templates for DNA or RNA synthesis by polymerases using techniques well known in the art, such as using the polymerase chain reaction. DNA synthesis may be primed using random primers. Random priming is described in U.S. Pat. Nos. 5,106,727 and 5,043,272. In specific embodiments, the DNA polymerase is not a thermostable DNA polymerase. In a specific embodiment, the DNA polymerase is selected from the group consisting of Klenow fragment of E. coli DNA polymerase I, reverse transcriptase, bacteriophage T7 DNA polymerase, bacteriophage φ29 DNA polymerase, Tts DNA polymerase, phage M2 DNA polymerase, VENT™ DNA polymerase, T5 DNA polymerase, PRD1 DNA polymerase, T4 DNA polymerase holoenzyme, T7 native polymerase T7 Sequenase®, or Bst DNA polymerase. In a preferred embodiment, the DNA polymerase is the Klenow fragment of E. coli DNA polymerase I. RNA polymerases may also be used to generate RNA probes.

In some embodiments, the labeled probes are generated using ligation-mediated polymerase chain reaction (LM-PCR). LM-PCR is described, for example, in U.S. application No. 2003/0143599. Other methods for DNA labeling include direct labeling, 77 RNA polymerase amplification, aminoallyl labeling and hapten-antibody enzymatic labeling. In one embodiment, the labeled probes comprise a flourescent molecule, such as Cy3 or Cy5 dyes. In another embodiment, the labeled probes comprise semiconducting nanocrystals, also known as quantum dots. Quantum dots are described in U.S. Publication Nos. 2003/0087239 and 2002/0028457, and in international PCT publication No. WO01/61040.

In one embodiment, identifying a region of the genome of the cell which is complementary to the isolated DNA fragments comprises combining the labeled probe with at least one nucleic acid comprising a sequence complementary to a region of the genome of the cell, under conditions in which hybridization between the labeled probe and the nucleic acid occurs, and detecting said hybridization, wherein hybridization between the labeled probe and the nucleic acid relative to a suitable control indicates that the protein of interest is bound to the region of the genome to which the sequence of the nucleic acid is complementary.

In one embodiment, the labelled or unlabeled probes are hybridized to DNA microarray, such as is described in U.S. Pat. No. 6,410,243, which microarrays comprise nucleic acids with sequences complementary to regions of the genome of the cell. Microarrays, also called “biochips” or “arrays” are miniaturized devices typically with dimensions in the micrometer to millimeter range for performing chemical and biochemical reactions and are particularly suited for embodiments of the invention. The microarrays may comprise nucleic acids complementary to the entire genome of the cell, to intergenic regions, intragenic regions, euchromatin regions, promoter regions or other known gene expression regulatory regions. In one embodiment, the microarray comprises a plurality of nucleic acids which are complementary to at least 1%, 2%, 3%, 4%, 5%, 10% or 50% of (i) the genome of the cell, (ii) the euchromatin of the cell, or (iii) the promoter regions of the cell.

Arrays may be constructed via microelectronic and/or microfabrication using essentially any and all techniques known and available in the semiconductor industry and/or in the biochemistry industry, provided only that such techniques are amenable to and compatible with the deposition and screening of polynucleotide sequences. Microarrays are particularly desirable for their virtues of high sample throughput and low cost for generating profiles and other data. A DNA microarray for use in the present invention may be constructed with spots that comprise nucleic acid with promoter sequences. Additional variations for manipulating and examining chromatin using microarrays have been described in U.S. Pat. Nos. 6,410,243, the teachings of which are incorporated herein by reference.

DNA microarray and methods of analyzing data from microarrays are well-described in the art, including in DNA Microarrays: A Molecular Cloning Manual, Ed by Bowtel and Sambrook (Cold Spring Harbor Laboratory Press, 2002); Microarrays for an Integrative Genomics by Kohana (MIT Press, 2002); A Biologist's Guide to Analysis of DNA Microarray Data, by Knudsen (Wiley, John & Sons, Incorporated, 2002); and DNA Microarrays: A Practical Approach, Vol. 205 by Schema (Oxford University Press, 1999); and Methods of Microarray Data Analysis II, ed by Lin et al. (Kluwer Academic Publishers, 2002), hereby incorporated by reference in their entirety.

In one embodiment of the methods described, labeled probes from control DNA fragments and labeled probes from the isolated DNA fragments are hybridized to a DNA microarray that includes experimental spots that represent all or a subset (e.g., a chromosome or chromosomes) of the genome. The fluorescent intensity of each experimental spot on the microarray from each of the labeled probes is determined, indicating whether the protein of interest is bound to the DNA region located at that particular spot. Hence, the methods described herein allow the detection of protein- DNA interactions across an entire genome or portions thereof. Suitable control probes may be generated from the mixture of DNA fragments of step (a).

In some embodiments, detecting the hybridization between the labeled/unlabeled probes and the nucleic acids complimentary to the genome is facilitated by contacting the complexes between the labeled or unlabeled probe and the nucleic acid with a detection agent, wherein the amount of detection agent that binds to the complex is indicative of the level of hybridization. In one embodiment, the detection agent comprises an antibody or fragment thereof. In another embodiment, the detection agent comprises a dendrimer. The use of dendrimers to for the detection microarray hydridization has been described in U.S. Patent Pub. Nos. 2002/0051981 and 2002/0072060, hereby incorporated by reference in their entirety. In another embodiment, the detection agent binds to a double stranded nucleic acid selected from the group consisting of a DNA-DNA, DNA-RNA or RNA-RNA double stranded-nucleic acids.

In another embodiment, identifying a region of the genome of the cell which is complementary to the isolated DNA fragments comprises PCR amplification of the isolated DNA fragment with oligonucleotide primers which specifically amplify a region of the genome, wherein a PCR product is generated if the isolated DNA fragment comprises said region of the genome. This approach may be preferable to the use of microarrays when few regions in the chromosome are of interest.

Protein of Interest

In some embodiments of the methods described herein, the protein of interest is native to the cell, whereas in other embodiments the protein of interest is a recombinant protein. By native it is meant that the protein of interest occurs naturally in the cell. In some embodiments, the protein of interest is a transcriptional regulator. In specific embodiments, the transcriptional regulator is a recombinant transcriptional regulator. In some embodiments, the transcriptional regulator originates from a species which is different from that of the cell. In some embodiments, the transcriptional regulator is a viral transcriptional regulator. In such embodiments, a cell may be contacted with a virus and chromatin extracted from the infected cell after allowing sufficient time for the viral proteins to be expressed. In some embodiments, recombinant transcriptional regulators have missense mutations, truncations, or inserted sequences or entire domains from other naturally occurring proteins. A tagged recombinant transcriptional regulator may be used in some embodiments as the tag may facilitate the immunoprecipitation of the regulator.

In certain embodiments of the invention, the protein of interest comprises specific transcription factors, coactivators, corepressors or complexes thereof. Transcription factors bind to specific cognate DNA elements such as promoters, enhancers and silencer elements, and are responsible for regulating gene expression. Transcription factors may be activators of transcription, repressors of transcription or both, depending on the cellular context. Transcription factors may belong to any class or type of known or identified transcription factor. Examples of known families or structurally-related transcription factors include helix-loop-helix, leucine zipper, zinc finger, ring finger, and hormone receptors. Transcription factors may also be selected based upon their known association with a disease or the regulation of one or more genes. For example, transcription factors such as c-myc, Rel/Nf-kB, neuroD, c-fos, c-jun, and E2F may be targeted. Antibodies directed to any transcriptional coactivator or corepressor may also be used according to the invention. Examples of specific coactivators include CBP, CTIIA, and SRA, while specific examples of corepressors include the mSin3 proteins, MITR, and LEUNIG. Furthermore, the genes regulated by proteins associated with transcriptional complexes, such as the histone acetylases (HATs) and histone deacetylases (HDACs), may also de determined using the methods described herein.

In some embodiments of the methods described herein, the cell has been treated with an agent, such as compound or a drug, prior to the fragmenting of genomic DNA and preferably while the cell is alive. Some preferred agents include those which bind to and/or regulate the expression of transcriptional regulators, or which are suspected of doing so. In some embodiments, the regions of the genome that are bound by a given transcriptional regulator are determined both in a cell that is contacted with an agent and in a cell that is not contacted with the agent, or that is contacted with a different amount of the agent. Such methods may be used to identify compounds that alter the types of genes and/or the extent to which a transcriptional regulators controls transcription of genes. Furthermore, such approaches may be used to screen for agents which alter the activity, DNA-binding specificity or expression of a transcriptional regulator.

In other embodiments of the methods described herein, the protein of interest is a DNA-binding protein, such as a basal transcription factor or a component of the basal transcription machinery. Exemplary components of the basal transcription machinery include RNA polymerases, including polI, polII and polIII, TBP, NTF-1 and Sp1 and any other component of TFIID, including, for example, the TAFs (e.g. TAF250, TAF150, TAF135, TAF95, TAF80, TAF55, TAF31, TAF28, and TAF20), or any other component of a polymerase holoenzyme. In one embodiment of the methods described above, the member of the transcriptional machinery is an RNA polymerase, such as RNA polymerase II, a TATA-binding protein, or any other component of TFIID, including, for example, the TAFs (e.g. TAF250, TAF150, TAF135, TAF95, TAF80, TAF55, TAF31, TAF28, and TAF20).

In specific embodiments of the methods described herein, the protein of interest is a transcription factor selected from the group consisting of SOX1-18, OCT6, PAX3, Myocardin, GATA1-6, TCF1/HNF1A, HNF4A, HNF6, NGN3, C/EBP, FOXA1-3, IPF1, GATA, HNF3, NKX2.1, CDX, FTF/NR5A2, C/EBPbeta, SCL1, SKIN1, or a member of the neurogenin, LK, LMO, SOX, OCT, PAX, GATA and the MyoD family of transcription factors.

In specific embodiments of the methods described herein, the protein of interest is a transcription factor selected from the group consisting of PAX3, EGR-1, EGR-2, OCT6, a SOX family member, a GATA family member, a PAX family member, an OCT family member, RFX5, WHN, GATA1, VDR, CRX, CBP, MeCP2, AML1, p53, PLZF, PML, Rb, WT1, NR3C2, GCCR, PPARgamma, SIM1, HNF1α, HNF1β, HNF4′, PDX1, MAFA, FOXA2, and NEUROD1.

The methods described herein may be applied to protein of interest that has been causally implicated in a disease. Examples of diseases and transcriptional regulators which cause them may be found in the scientific and medical literature by one skilled in the art, including in Medical Genetics, L. V. Jorde et al., Elsevier Science 2003, and Principles of Internal Medicine, 15th edition, ed by Braunwald et al., McGraw-Hill, 2001; American Medical Association Complete Medical Encyclopedia (Random House, Incorporated, 2003); and The Mosby Medical Encyclopedia, ed by Glanze (Plume, 1991). In some embodiments, the disorder is characterized by impaired function of at least one of the following organs or tissues: brain, spinal cord, heart, arteries, esophagus, stomach, small intestine, large intestine, liver, pancreas, lungs, kidney, urinary tract, ovaries, breasts, uterus, testis, penis, colon, prostate, bone, scalp, muscle, cartilage, thyroid gland, adrenal gland, pituitary, bone marrow, blood, thymus, spleen, lymph nodes, skin, eye, ear, nose, teeth or tongue.

Plates and Microfluidics

In preferred embodiments of the methods described herein, the cell populations are contained within wells of multi-well plates to facilitate parallel handling of cells and reagents. In specific embodiments, the multi-well plate has 24, 48, 96 or 384 wells. Standard 96 well microtiter plates which are 86 mm by 129 mm, with 6 mm diameter wells on a 9 mm pitch, may be used for compatibility with current automated loading and robotic handling systems. The microplate is typically 20 mm by 30 mm, with cell locations that are 100-200 microns in dimension on a pitch of about 500 microns. Methods for making microplates are described in U.S. Pat. No. 6,103,479, incorporated by reference herein in its entirety.

Microplates may consist of coplanar layers of materials to which cells adhere, patterned with materials to which cells will not adhere, or etched 3-dimensional surfaces of similarly pattered materials. For the purpose of the following discussion, the terms “well” and “microwell” refer to a location in an array of any construction to which cells adhere and within which the cells are imaged. Microplates may also include fluid delivery channels in the spaces between the wells. The smaller format of a microplate increases the overall efficiency of the system by minimizing the quantities of the reagents, storage and handling during preparation and the overall movement required for the scanning operation. In addition, the whole area of the microplate can be imaged more efficiently. Multi-well test plates used for isotopic and non-isotopic assays are well known in the art and are exemplified, for example, by those described in U.S. Pat. Nos. 3,111,489; 3,540,856; 3,540,857; 3,540,858; 4,304,865; 4,948,442; and 5,047,215.

Microfluidic devices may also be used at any of the steps of the high-throughput methods described herein. For example, Chung et al. (2004) Lab Chip.;4(2):141-7 describe a high efficiency DNA extraction microchip was designed to extract DNA from lysed cells using immobilized beads and shaking solution, which allows extraction of as little as 10³ cells. Guijt et al. (2003) Lab Chip;3(1):1-4 describes microfluidic devices with accurate temperature control, as might be used to cycle temperature during PCR amplification. Similarly, Liu et al. (2002) Electrophoresis.;23(10):1531-6 teaches a microfluidic device for performing PCR amplification using as little as 12 nL of sample. Cady et al. (2003) Biosens Bioelectron. 30;19(1):59-66 describes a microfluidic device that may be used to purify DNA.

### IV. SCREENING EXPERIMENTAL AGENTS

In yet another specific embodiment, a suitable control comprises a measure binding in a population of cells that have not been contacted with the experimental agent; (b) have been contacted with a different dosage of the experimental agent; (c) have been contacted with a second experimental agent; or (d) a combination thereof.

In some embodiments of the methods described herein, the experimental agent comprises a small molecule drug, an antisense nucleic acid, an antibody, a peptide, a ligand, a fatty acid, a hormone or a metabolite.

Exemplary compounds that may be used as experimental agents (e.g., a single compound, a combination of two or more compounds, a library of compounds) include nucleic acids, peptides, polypeptides, peptidomimetics, antibodies, antisense oligonucleotides, RNAi constructs (including siRNAs), ribozymes, chemical compounds, and small organic molecules. Compounds may be screened individually, in combination, or as a library of compounds. Without being bound by theory, the invention contemplates that the modulation of cellular phenotypes may involve the activation or inhibition of particular genes and signaling pathways which modulate proliferation, survival, or differentiation along a particular lineage, thereby modulating a cellular phenotype.

Test compounds can be screened individually, in combination with one or more other compounds, or as a library of compounds. Compounds include nucleic acids, peptides, polypeptides, peptidomimetics, RNAi constructs, antisense oligonucleotides, ribozymes, antibodies, and small molecules. Numerous mechanisms exist to promote or inhibit the expression and/or activity of a particular mRNA or protein. The following are illustrative examples of exemplary classes of compounds that promote or inhibit expression and/or activity of nucleic acids or proteins or that promote or inhibit signal transduction via a signaling pathway, which may be used as experimental agents. Such compounds can be screened according to the methods of the present invention to identify and/or characterize compounds that modulate the binding of a protein to DNA.

Antisense oligonucleotides are relatively short nucleic acids that are complementary (or antisense) to the coding strand (sense strand) of the mRNA encoding a particular protein. Although antisense oligonucleotides are typically RNA based, they can also be DNA based. Additionally, antisense oligonucleotides are often modified to increase their stability.

Without being bound by theory, the binding of these relatively short oligonucleotides to the mRNA is believed to induce stretches of double stranded RNA that trigger degradation of the messages by endogenous RNases. Additionally, sometimes the oligonucleotides are specifically designed to bind near the promoter of the message, and under these circumstances, the antisense oligonucleotides may additionally interfere with translation of the message. Regardless of the specific mechanism by which antisense oligonucleotides function, their administration to a cell or tissue allows the degradation of the mRNA encoding a specific protein. Accordingly, antisense oligonucleotides decrease the expression and/or activity of a particular protein.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or compounds facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-floroarabinose, xylulose, and hexose. The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an -anomeric oligonucleotide. An -anomeric oligonucleotide forms specific double-stranded hybrids with complementary RNA in which, contrary to the usual-units, the strands run parallel to each other (Gautier et al., 1987, Nucl. Acids Res. 15:6625-6641). The oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

The selection of an appropriate oligonucleotide can be readily performed by one of skill in the art. Given the nucleic acid sequence encoding a particular protein, one of skill in the art can design antisense oligonucleotides that bind to that protein, and test these oligonucleotides in an in vitro or in vivo system to confirm that they bind to and mediate the degradation of the mRNA encoding the particular protein. To design an antisense oligonucleotide that specifically binds to and mediates the degradation of a particular protein, it is important that the sequence recognized by the oligonucleotide is unique or substantially unique to that particular protein. For example, sequences that are frequently repeated across protein may not be an ideal choice for the design of an oligonucleotide that specifically recognizes and degrades a particular message. One of skill in the art can design an oligonucleotide, and compare the sequence of that oligonucleotide to nucleic acid sequences that are deposited in publicly available databases to confirm that the sequence is specific or substantially specific for a particular protein.

In another example, it may be desirable to design an antisense oligonucleotide that binds to and mediates the degradation of more than one message. In one example, the messages may encode related protein such as isoforms or functionally redundant protein. In such a case, one of skill in the art can align the nucleic acid sequences that encode these related proteins, and design an oligonucleotide that recognizes both messages.

A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous mRNAs in certain instances. Therefore another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells.

Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo.

As used herein, the term “dsRNA” refers to siRNA molecules, or other RNA molecules including a double stranded feature and able to be processed to siRNA in cells, such as hairpin RNA moieties.

The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response, e.g., a PKR response.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci U S A, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO01/68836 and WO01/75164.

Ribozyme molecules designed to catalytically cleave an mRNA transcript can also be used to prevent translation of mRNA (See, e.g., PCT International Publication WO90/11364, published Oct. 4, 1990; Sarver et al., 1990, Science 247:1222-1225 and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216). The Cech-type ribozymes have an eight base pair active site that hybridizes to a target RNA sequence whereafter cleavage of the target RNA takes place. The invention encompasses those Cech-type ribozymes that target eight base-pair active site sequences.

As in the antisense approach, the ribozymes can be composed of modified oligonucleotides (e.g., for improved stability, targeting, etc.) and can be delivered to cells in vitro or in vivo. A preferred method of delivery involves using a DNA construct “encoding” the ribozyme under the control of a strong constitutive pol III or pol II promoter, so that transfected cells will produce sufficient quantities of the ribozyme to destroy targeted messages and inhibit translation. Because ribozymes unlike antisense molecules, are catalytic, a lower intracellular concentration is required for efficiency.

Antibodies can be used as inhibitors of the activity of a particular protein. Antibodies can have extraordinary affinity and specificity for particular epitopes. Antibodies that bind to a particular protein in such a way that the binding of the antibody to the epitope on the protein can interfere with the function of that protein. For example, an antibody may inhibit the function of the protein by sterically hindering the proper protein-protein interactions or occupying active sites. Alternatively the binding of the antibody to an epitope on the particular protein may alter the conformation of that protein such that it is no longer able to properly function.

Monoclonal or polyclonal antibodies can be made using standard protocols (See, for example, Antibodies: A Laboratory Manual ed. by Harlow and Lane (Cold Spring Harbor Press: 1988)). A mammal, such as a mouse, a hamster, a rat, a goat, or a rabbit can be immunized with an immunogenic form of the peptide. Techniques for conferring immunogenicity on a protein or peptide include conjugation to carriers or other techniques well known in the art.

Following immunization of an animal with an antigenic preparation of a polypeptide, antisera can be obtained and, if desired, polyclonal antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a particular polypeptide and monoclonal antibodies isolated from a culture comprising such hybridoma cells.

The term antibody as used herein is intended to include fragments thereof which are also specifically reactive with a particular polypeptide. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific and chimeric molecules having affinity for a particular protein conferred by at least one CDR region of the antibody.

Both monoclonal and polyclonal antibodies (Ab) directed against a particular polypeptides, and antibody fragments such as Fab, F(ab)₂, Fv and scFv can be used to block the action of a particular protein. Such antibodies can be used either in an experimental context to further understand the role of a particular protein in a biological process, or in a therapeutic context.

Peptides, polypeptides, variants polypeptides, and peptide fragments can be test compounds. Exemplary polypeptides comprise an amino acid sequence at least 60%, 70%, 75%, 80%, 85%, 90%, 95%, 98%, 99%, or 100% identical to a particular polypeptide. Exemplary fragments include fragments of at least 5, 6, 7, 8, 9, 10, 15, 20, 25, 50, 75, 100, 125, 150, 200, 250, or greater than 250 amino acid residues of the full length polypeptide. Peptides and polypeptides can either agonize or antagonize the function of a particular protein, and thereby modulate the phenotype of a cell.

Small organic molecules can either agonize or antagonize the expression and/or activity of a particular protein, and thereby modulate the phenotype of a cell. By small organic molecule is meant a carbon contain molecule having a molecular weight less than 2500 amu, more preferably less than 1500 amu, and even more preferably less than 750 amu. In the context of the present invention, such small organic molecules would be able to promote the differentiation of a cell to a particular differentiated cell type.

Small molecules can be readily identified by screening libraries of organic molecules and/or chemical compounds to identify those compounds that have a desired function. Without being bound by theory, small organic molecules may influence a cellular phenotype in any of a number of ways. By way of example, small molecules may act at the cell surface to influence cell surface receptors. By way of further example, small molecules may act intracellularly to influence intracellular signaling along a particular signaling pathway. The methods of the present invention are unbiased and allow identification of small molecule compounds that modulate a cellular phenotype regardless of its mechanism of action.

In addition to compounds which are peptides or polypeptides, the invention contemplates nucleic acids comprising nucleotide sequences encoding peptides and polypeptides. The term nucleic acid as used herein is intended to include equivalents. The term equivalent is understood to include nucleotide sequences which are functionally equivalent to a particular nucleotide sequence. Equivalent nucleotide sequences will include sequences that differ by one or more nucleotide substitutions, additions or deletions, such as allelic variants, and variation due to degeneracy of the genetic code. Equivalent sequences may also include nucleotide sequences that hybridize under stringent conditions (i.e., equivalent to about 20-27° C. below the melting temperature (Tₘ) of the DNA duplex formed in about 1M salt) to a given nucleotide sequence. Further examples of stringent hybridization conditions include a wash step of 0.2×SSC at 65° C.

Nucleic acids having a sequence that differs from nucleotide sequences which encode a particular peptide or polypeptide test compound due to degeneracy in the genetic code are also within the scope of the invention. Such nucleic acids encode functionally equivalent peptides but differ in sequence from wildtype sequences known in the art due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence. However, it is expected that DNA sequence polymorphisms that do lead to changes in the amino acid sequences will also exist.

Biological conditions include any biological aspect of the shared fluid volume in which our cell populations are disposed. The biological aspects may include the presence, absence, concentration, activity, or type of cells, viruses, vesicles, organelles, biological extracts, and/or biological mixtures, among others. The assays described herein may screen a library of conditions to test the activity of each library member on a set of cell populations. A library generally comprises a collection of two or more different members. These members may be chemical modulators (or candidate modulators) in the form of molecules, ligands, compounds, transfection materials, receptors, antibodies, and/or cells (phages, viruses, whole cells, tissues, and/or cell extracts), among others, related by any suitable or desired common characteristic. This common characteristic may be “type.” Thus, the library may comprise a collection of two or more compounds, two or more different cells, two or more different antibodies, two or more different nucleic acids, two or more different ligands, two or more different receptors, or two or more different phages or whole cell populations distinguished by expressing different proteins, among others. This common characteristic also may be “function.” Thus, the library may comprise a collection of two or more binding partners (e.g., ligands and/or receptors), agonists, or antagonists, among others, independent of type.

Library members may be produced and/or otherwise generated or collected by any suitable mechanism, including chemical synthesis in vitro, enzymatic synthesis in vitro, and/or biosynthesis in a cell or organism. Chemically and/or enzymatically synthesized libraries may include libraries of compounds, such as synthetic oligonucleotides (DNA, RNA, peptide nucleic acids, and/or mixtures or modified derivatives thereof), small molecules (about 100 Da to 10 KDa), peptides, carbohydrates, lipids, and/or so on. Such chemically and/or enzymatically synthesized libraries may be formed by directed synthesis of individual library members, combinatorial synthesis of sets of library members, and/or random synthetic approaches. Library members produced by biosynthesis may include libraries of plasmids, complementary DNAs, genomic DNAs, RNAs, viruses, phages, cells, proteins, peptides, carbohydrates, lipids, extracellular matrices, cell lysates, cell mixtures, and/or materials secreted from cells, among others. Library members may be contact arrays of cell populations singly or as groups/pools of two or more members.

### V. KITS

Some aspects of the invention provides kits for performing high-throughput genome-wide location analysis, or for performing one or two of the such methods. One aspect of the invention provides a kit comprising (i) one or more DNA endonucleases; and (ii) a dilution buffer lacking free amino groups. In some embodiments. In one embodiment, the one or more DNA endonuclease has an optimal temperature for enzymatic cleavage of DNA lower than 15%, 20° C., 25° C., 30° C., 35° C., or 37° C. In a specific embodiment, the endonuclease retains at least 5%, 10%, 15%, 20%, 25% 30%, 40%, 50%, 60%, 70%, 80%, 90% or 95% of its maximum enzymatic activity at a temperature of 25° C, 20° C., 15° C., 10° C., 4° C. or 0° C., or at about room temperature.

In one embodiment, the endonuclease is DNAse I. In another embodiment, the endonuclease is a restriction enzyme endonuclease. In some embodiments, the restriction endonucleases recognize 4, 5, 6, 7 or 8 base pair recognition sequences. In some embodiments, the kit comprises 2, 3, 4, 5, 6, 7, 8, 9 10 or more restriction enzyme endonuclease, and optionally DNAseI. In one embodiment, the restriction enzyme endonuclease(s) is selected from Sfu I (Asu II), Afl III, Bfr I (Afl II), BbrP I (PmaC I), BssH II, Eco47 III, Ecl XI (Xma III), Hind III, Mam I, Nsp I, Ksp I (Sac II), BstX I, Ita I, Mro I (Acc III), Nar I, Cel II, Cfr10 I, Hind II, Avi II (Aos I), ScrF I (Dsa V), Acy I (Aha II), AspH I (HgiA I), Ksp632 I, Mvn I (FnuD II), Eae I (Cfr I), Sty I, Nde I, EcoR I, Nde II (Mbo I), Not I, Spe I, Fok I, SnaB I, Ssp I, Nsi I, Mlu I, Nhe I, Mae II, Cla I, Dra III, Dra II, Nco I, Dde I, Asp700 (Xmn I), Mae III, Mae I, Asp718, Nae I, Dra I, BstE II, Hinf I, Nru I, Sca I, BpuA I, Aat II, Ban II, Stu I, Dpn I, Bgl II, Xho II, Kpn I, Ava II, Ava I, SspB I, Rsa I, Acc I, Xho I, Apa I, Bgl I, Sau3A I, Hae III, Hae II, Bcl I, Cfo I (Hha I), Xba I, Sac I (Sst I), EcoR V, Sau96 I, BamH I, Pvu I, Pvu II, Msp I, Sph I, Taq I, Sma I, Sal I, XmaC I, BspLU11 I, Bln I (Avr II), Psp1406 I, Acs I, MluN I (Bal I), SexA I, Rca I (BspH I), PinA I (Age I), Pst I, Tru9 I, Alw44 I (Sno 1), Mun I (Mfe I), AspE I, EcoR II (BstN I), BseA I, BsiW I, BsiY I, Van91 I (PflM I), Hpa II, Bst1 107 I, Swa I, Meganuclease I-Sce I, Omega Nuclease Omega Transposase, Rsr II, Bsm I, Mva I (BstN I), Sfi I, SgrA I, Bmy I (Bsp1286 I), Hpa I and Alu I. In certain embodiments, the restriction enzyme endonuclease(s) is selected from the group consisting of Sau3a, Styl, NlaIII and Hsp 92.

In one embodiment, the kits comprise a dilution buffer for performing enzymatic reactions with the endonuclease. The dilution buffer may be in concentrated form, such as 10 or 100-fold concentrated, for proper dilution of a composition comprising the endonuclease. In one preferred embodiment, the dilution buffer does not have free amino groups. In another embodiment, it is substantially free of tris(hydroxymethyl) aminomethane. In certain embodiments, the concentration of tris, or of free amino groups, in the dilution buffer is less than 5 mM, 3mM, 1 mM, 0.5 mM, 0.3 mM, 0.1 mM, 0.05 mM, 0.01 mM, or 0.001 mM. In one embodiment, the dilution buffer comprises a buffering agent selected from alkali or alkali earth tartrates, alkali or alkali earth carbonates, phosphates, bicarbonates, citrates, borates, acetates, and succinates. In another embodiment, the dilution buffer comprises a buffering agent selected from the group consisting of HEPES (N-2-hydroxyethylpiperazine-N′-2-ethanesulfonic acid), BES (N,N-bis[2-hydroxyethyl]-2-amino-ethanesulfonic acid), TES (N-tris[Hydroxymethyl]methyl-2-aminoethanesulfonic acid), MOPS (morpholine propanesulphonic acid), PIPES (piperazine-N,N′-bis[2-ethane-sulfonic acid]) and MES (2-morpholino ethanesulphonic acid). In another embodiment, the buffering agent sodium citrate/citric acid buffers or phosphate buffers. In some embodiments, the dilution buffer contains more than one buffering agent.

In one embodiment, the kits further comprise one or more multiwell-filter plates suitable for purification of DNA. In another embodiment, the kit further comprises a manganese porphyrin complex, such as Mn-TMPyP/KHSO₅. (see (Chworos A et al. J Biol Inorg Chem. (2004);9(3):374-84)).

In another embodiment, the kit comprises a protease. In one embodiment, the protease is an aspartic protease, serine protease, thiol protease, metallo protease, acid protease or an alkaline protease. In another embodiment, the protease is a serine protease such as thrombin, plasmin, factor Xa, uPA, tPA, granzyme B, trypsin, chymotrypsin, human neutrophil elastase, or a cysteine protease such as papain and cruzain. In a preferred embodiment, the protease is proteinase K. A mixture of proteases may also be included in the kit, either mixed together in separate containers.

In some embodiments, the kits further comprise one or more RNA-degrading enzymes (RNase), such as an RNase exonuclease. In some embodiments, the RNase is an endonuclease such as RNase E. In another embodiment, the RNase is selected from RNase A, RNase H, RNase One, RNase B, RNase T1, RNase T2, RNase S, RNase from chicken liver, RNase from Aspergillus clavatus, and pancreatic RNase.

In another embodiment, the kits also include packaging material such as, but not limited to, ice, dry ice, styrofoam, foam, plastic, cellophane, shrink wrap, bubble wrap, paper, cardboard, starch peanuts, twist ties, metal clips, metal cans, drierite, glass, and rubber.

### EXEMPLIFICATION

The invention now being generally described, it will be more readily understood by reference to the following examples, which are included merely for purposes of illustration of certain aspects and embodiments of the present invention, and are not intended to limit the invention, as one skilled in the art would recognize from the teachings hereinabove and the following examples, that other DNA microarrays, transcriptional regulators, cell types, antibodies, ChIP conditions, or data analysis methods, all without limitation, can be employed, without departing from the scope of the invention as claimed.

The practice of the present invention will employ, where appropriate and unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999; and PCR Protocols, ed. by Bartlett et al., Humana Press, 2003.

Various publications, patents, and patent publications are cited throughout this application the contents of which are incorporated herein by reference in their entirety.

### Example 1

This is a sample protocol for generating labeled probes from yeast.

Preparation of Cells and Crosslinking

Inoculate fresh media from an overnight culture to OD₆₀₀=0.1 and allow yeast to grow to OD₆₀₀=0.6-1.0 (I use OD₆₀₀ 0.8). Remove 50 ml cells and add to 50 ml Falcon tubes containing 1.4 ml of Formaldehyde (37% Formaldehyde stock, final concentration 1%). Incubate for 20 minutes at room temperature on a rotating wheel. Spin 50 ml Falcon tubes for 5 minutes at 2800 rpm in a tabletop centrifuge to harvest the cells and pour off the supernatant. Wash cells. Add TBS, mix by inversion until the cells are resuspended, spin and pour off the supernatant. Wash 2× more. After pouring off the supernatant from the last wash, resuspend the yeast pellet using remaining liquid and transfer to 96-well plate. Spin for 1 minute. at maximum speed at 4° C. and remove the remaining supernatant. Store pellets at −80° C., or go directly to step 2.

Cell Lysis

Thaw cell pellet briefly on ice. Resuspend in 700 μl of 1× enzyme digestion buffer. This is a HEPES based buffer. If necessary, transfer cells to consolidate into fewer plates. Add the equivalent of a 0.5 ml PCR of glass beads (425-600 μm, Sigma Cat.# G-8772). Shake for 15 minutes at 4° C. using tabletop 96-well plate shaker. Using 96-well plate filter system, remove beads and transfer sample to new 96-well plate. Sonicate using Covaris sonication system to solubilize chromatin. Clarify extracts by centrifugation. Add 75 units restriction enzyme cocktail and digest overnight at 16° C. Set aside 10 μl of each extract for use as input control. Transfer 400 μl of each extract to new plate. Add 400 μl of lysis buffer. Add 60 μl of a suspension of prepared magnetic beads coated with an antibody which binds a protein of interest. Cover plate and incubate overnight at 4° C. with rotation.

Bead Washing

All bead washing is done at 4° C. Wash beads using appropriate device (e.g. MPC-E magnet, Dynal) Wash 2 times with 1 ml lysis buffer. Wash 2 times with 1 ml lysis buffer containing 360 mM NaCl (72 μl of 5M NaCl in 10 ml lysis buffer). Wash 2 times with 1 ml wash buffer. Wash once with 1 ml TE. Remove as much TE as you can by aspiration.

Elution from Beads and Reversal of Cross Links

Add 50 μl elution buffer to beads, vortex briefly to resuspend the beads and incubate at 65° C. overnight. Remove the plate with 10 μl of WCE from the −80° C. freezer and add 40 μl of elution buffer to each well. Incubate at 65° C. overnight.

Precipitation of DNA

Remove plates from 65° C. incubator. Transfer supernatants to new plates. To each well, add 400 μl of TE+RNase A (4 μl of 10 mg/ml RNase A). Incubate for 2 hours at 37° C. in the warm room. Add 10 μl of proteinase K (20 mg/ml stock) and 4 μl of glycogen (10 mg/ml stock). Incubate for 2 hours at 37° C. in the warm room. Clean up DNA using 96-well format cleanup kit.

Labeling DNA

Transfer 44 μl of IP DNA to 96-well plates suitable for PCR. Transfer 2 μl of corresponding WCE DNA. Add 42 μl of water to WCE DNA. Add 40 μl of 2.5× Random Primers Solution to all wells. Denature DNA (10 minutes at 95° C.) and immediately cool on ice. Add: 10 μl of 10× dNTP mix, 4 μl of Cy3 or Cy5 dye, 2 μl of Klenow fragment. Mix gently with pipetting. Incubate at 37° C. overnight. Add 10 μl stop buffer Clean up using 96-well format cleanup kit (preferable spin column).

1. A method for identifying a region of a genome of a cell to which a protein of interest is bound, the method comprising the steps of:
(a) fragmenting the genomic DNA of the cell by:
(i) a mechanical or chemical process; and
(ii) by an enzymatic means, thereby producing a mixture comprising DNA fragments to which the protein of interest is bound;
(b) isolating a DNA fragment to which the protein of interest is bound from the mixture produced in step (a); and
(c) identifying a region of the genome of the cell which is complementary to the DNA fragment isolated in step (b),
thereby identifying a region of a genome of a cell to which the protein of interest is bound.

2. The method of claim 1, wherein step (c) comprises generating a labeled probe from the DNA fragment isolated in step (b).

3. The method of claim 2, wherein step (c) comprises combining the labeled probe with at least one nucleic acid comprising a sequence complementary to a region of the genome of the cell, under conditions in which hybridization between the labeled probe and the nucleic acid occurs, and detecting said hybridization, wherein hybridization between the labeled probe and the nucleic acid relative to a suitable control indicates that the protein of interest is bound to the region of the genome to which the sequence of the nucleic acid is complementary.

4-18. (canceled)

19. The method of claim 1, wherein the protein of interest is covalently crosslinked to the genomic DNA prior to fragmenting the genomic DNA of the cell.

20-21. (canceled)

22. The method of claim 21, wherein isolating the DNA fragment from the protein of interest to which it is bound comprises the steps of
(1) removing the crosslink between the DNA fragment and the protein of interest;
(2) treating the DNA fragment with an RNA-degrading enzyme;
(3) treating the DNA fragment with a protease; and
(4) purifying the DNA fragment.

23-44. (canceled)

45. The method of claim 1, wherein the enzymatic means comprises an endonuclease.

46-50. (canceled)

51. The method of claim 45, wherein the fragmentation by enzymatic means is performed at a temperature below the optimum temperature for endonuclease catalysis.

52. The method of claim 51, wherein the optimum temperature is about 37° C.

53. The method of claim 49, wherein the fragmentation by enzymatic means is performed at a temperature below 25° C.

54. The method of claim 1, wherein fragmenting by enzymatic means is performed in a solution comprising a buffering agent.

55. The method of claim 51, wherein the buffering agent is not tris(hydroxymethyl) aminomethane.

56. The method of claim 51, wherein the buffering agent does not contain free amino groups.

57. The method of claim 1, wherein fragmenting by enzymatic means is performed in a solution substantially free of tris(hydroxymethyl) aminomethane.

58. The method of claim 1, wherein fragmenting the genomic DNA with the mechanical or chemical means generates DNA fragments having an average size of 2 kb or greater.

59-62. (canceled)

63. The method of claim 1, wherein the chemical process comprises a manganese porphyrin complex.

64. (canceled)

65. The method of claim 1, wherein the chemical process comprises acid catalytic hydrolysis, alkaline catalytic hydrolysis, hydrolysis by metal ions, hydroxyl radicals or irradiation.

66-73. (canceled)

74. A method of identifying the region of a genome of a stem cell to which a protein of interest is bound during differentiation of the stem cell, the method comprising
(a) culturing the cell under conditions that promote the differentiation of the cell; and
(b) identifying the region of a genome of a stem cell to which a protein of interest is bound, according to the method of claim 1.

75. (canceled)

76. A method of screening a panel of mutant transcriptional regulators for regulators which bind to a specific set of regions of a genome in a cell, the method comprising
(a) expressing each transcriptional regulator in a cell;
(b) identifying regions of the genome of the cell to which each transcriptional regulator binds, according to the method of claim 1;
(c) comparing the regions of the genome of the cell to which each transcriptional regulator binds to the specific set of regions and selecting those transcriptional regulators which bind to the specific set of regions.

77. A high-throughput method of screening experimental agents for their ability to modulate the binding of a protein of interest to regions of a genome in a cell, the method comprising
(a) providing a plurality of samples each comprising a population of cells;
(b) contacting each sample with an experimental agent;
(c) identifying regions of the genome of the cells to which each protein of interest binds according to the method of claim 1; and
(d) comparing the identified regions to a suitable control.

78-83. (canceled)

84. A kit comprising (i) one or more DNA endonucleases; and (ii) a dilution buffer lacking free amino groups.

85-93. (canceled)
